(19) United States
(12) Patent Application Publication
(10) Pub. No.: US 2018/0147405 A1
(43) Pub. Date: May 31, 2018

(54) A SURGICAL IMPLANT CONDUCTOR WITH INCREASED RADIO FREQUENCY ALTERNATING CURRENT RESISTANCE

(71) Applicant: University of Waikato, Hamilton (NZ)

(72) Inventors: Steven Owen MCCABE, Porirua (NZ); Jonathan Brereton SCOTT, Hamilton (NZ)

(21) Appl. No.: 15/577,317

(22) PCT Filed: May 27, 2016
(86) PCT No.: PCT/NZ2016/050089

§ 371 Date: Nov. 27, 2017

(30) Foreign Application Priority Data
May 29, 2015 (NZ) 708633

(51) Int. Cl. A61N 1/05; H01J 37/32; C25F 3/02; A61N 1/08

(52) U.S. Cl. CPC A61N 1/05; H01J 37/32009; H01J 2237/334; A61N 1/086; C25F 3/02

(57) ABSTRACT

In one aspect the invention provides a surgical implant conductor formed from a length of conductive material which exhibits increased radio frequency alternating current resistance. This conductive material defines an exterior surface where at least a portion of the exterior surface of the conductor defines a region with a roughened surface. Preferably the roughening of the exterior surface increases the area of the exterior surface when compared to a non-roughened surface, resulting in a reduction in the effective cross-section area of the conductor used to transport alternating currents.

### FIELD OF THE INVENTION

This invention relates to a surgical implant conductor with increased radio frequency alternating current resistance. Preferably the invention may be used to increase the radio frequency alternating current resistance of a surgical implant conductor without significantly increasing the direct current resistance of this conductor.

### BACKGROUND OF THE INVENTION

A variety of therapeutic devices have been developed to deliver bioelectrical stimulation therapies. These devices are surgically implanted into the body of a patient and normally incorporate control electronics connected to a power supply system such as a battery pack. As these components can be relatively large they may be sited within a user's body some distance from an organ or tissue requiring bioelectrical stimulation. A surgical implant conductor or lead commonly needs to be run through the body from the power and control electronics to the organ requiring bioelectrical stimulation.

Surgical implant manufacturers select implant conductors which exhibit low electrical resistance to direct currents. This selection minimises the voltage required to generate therapeutic currents to maximize implant battery lifespan.

However, the presence of surgical implants creates a patient safety issue with the use of Magnetic Resonance Imaging (MRI) machines. MRI scanning machines expose patients to strong radio frequency electric fields in addition to strong magnetic fields. MRI machines are carefully designed and used under strict operational protocols to avoid inadvertent heating of patient body tissues.

Surgically implanted conductors act as antennas within the body and concentrate the strong radio frequency fields generated by an MRI machine. This is a significant patient safety problem when the length of the implant conductor is approximately equal to an odd integer multiple of the half wavelength of the electric field frequency—for example—λ/2, 3λ/2, 5λ/2 etc.

These safety issues normally inhibit patients with surgical implants from having access to full MRI scanning technology. It would therefore be of advantage to have available an improved implant conductor technology which addressed or mitigated the above problems, or at the very least provided an alternative choice over the current prior art. It would be of particular advantage to have a surgical implant conductor with improved radio frequency properties targeted at increasing the radio frequency alternating current resistance of the conductor without significantly increasing the direct current resistance of the conductor.

### DISCLOSURE OF THE INVENTION

According to one aspect of the present invention there is provided a surgical implant conductor with increased radio frequency alternating current resistance formed from a length of conductive material which defines an exterior surface wherein at least a portion of the exterior surface of the conductor defines a region with a roughened surface.

According to one aspect of the present invention there is provided a surgical implant conductor substantially as described above wherein the roughening of the exterior surface increases the area of the exterior surface when compared to a non-roughened surface.

According to a further aspect of the invention there is provided a method of manufacturing a surgical implant conductor substantially as described above characterised by the step of roughening at least a portion of the exterior surface of the conductor to define at least one region with a roughened surface.

Preferably the conductor may have a substantially circular cross-section profile which in various embodiments may extend the length of the conductor.

Preferably a roughened region of the exterior surface results in an increase of at least two times the surface area of the exterior surface when compared with a non-roughened surface.

Preferably a roughened region of the exterior surface results in an increase of between five to ten times the surface area of the exterior surface when compared with a non-roughened surface.

Preferably a roughened region defines at least one fissure extending into the body of the conductor.

Preferably a fissure extends into the body of the conductor with a substantially radial or transverse orientation.

Preferably a fissure formed in a roughened region extends into the body of the conductor to a depth approximately equal to or greater than the electrical skin depth of the conductor at the frequency of operation of the MRI machine.

The present invention is arranged to provide a surgical implant conductor for use in combination with surgical implant technology. Reference in general will be made throughout this specification to this implant conductor being used with a bioelectrical stimulation implant. In these applications a surgical implant conductor can be formed from a length of conductive material which has one end defining an electrode and an opposite end defining a supply terminal.

Those skilled in the art will however appreciate that the implant conductor provided by the invention may be used in other applications if required. In particular the invention may be used with implants which need not necessarily provide a bioelectrical stimulation effect nor provide a surgical implant conductor with one end forming an electrode or alternatively a supply terminal.

Furthermore the invention may potentially be used in combination with surgical implants installed in both humans and/or in animals. Those skilled in the art will appreciate that references made to implant technology does not restrict the use of the present invention to human recipients only.

A surgical implant conductor provided by the invention aims to provide improvements in the radio frequency alternating current handling characteristics of this conductor. Particularly the invention aims to increase the resistance experienced by radio frequency alternating currents travelling in the conductor without unduly increasing the direct current resistance of the conductor.

The implant conductor may be formed from materials currently employed as existing prior art implant conductors. In various embodiments the conductive material selected may exhibit desirable electrical characteristics while minimising patient tissue rejection or irritation effects.

In various embodiments the implant conductor may be formed from or composed of a number of different types of materials or elements. For example in some embodiments a surgical implant conductor may be composed from a core material surrounded and enclosed by a different surface material. These composite materials may allow electron transport and current flow over their boundary interfaces, with preferably the core material being selected on the basis of material cost and electrical characteristics, with the surface material being selected for its tissue rejection characteristics.

Reference in general will however be made throughout this specification to the conductive material employed by the invention being provided by a single type of material. However those skilled in the art will appreciate that prior art forms of composite implant lead materials may also be utilised in conjunction with the present invention.

The invention provides a surgical implant conductor formed from a length of conductive material. This conductor will therefore have a longitudinal length dimension which is substantially greater than its cross-section width. The implant conductor will define an exterior surface which extends between two ends of the length of conductive material.

Reference throughout this specification will also be made to the implant conductor being provided with two ends only. Again however, those skilled in the art will appreciate that other physical arrangements are also within the scope of the invention.

In preferred embodiments the conductor may be covered by an insulator which encloses the conductor while exposing the ends of the conductor. This insulating enclosure may be formed by any material which prevents therapeutic currents from escaping from the conductor other than at the ends of the conductor.

In a preferred embodiment the implant conductor may exhibit a substantially uniform transverse cross-section profile along its entire length. Reference throughout this specification will also be made to the implant conductor being provided with this uniform character, although those skilled in the art will appreciate that other arrangements and physical dimensions are also within the scope of the invention.

The present invention provides a surgical implant conductor where at least a portion of the exterior surface of this conductor defines a region with a roughened surface. This roughened region or regions provide the conductor with the desired electrical properties required of the invention.

In preferred embodiments this roughened surface region or regions may be covered by an insulating enclosure applied to the conductor.

In a preferred embodiment the entire exterior surface of the implant conductor may define a single region with a roughened surface.

In an alternative embodiments a roughened surface region may be located at or adjacent to the midpoint of the length of the conductor.

In some alternative embodiments the conductor may include a plurality of roughened surface regions which are discontinuous and located in discrete regions along the length of the exterior surface. Those skilled in the art will appreciate that higher proportions of roughened exterior surface to non-roughened surface will result in increased resistance to alternating currents.

In some embodiments at least 50% of the exterior surface of the conductor defines a roughened surface region or regions. In yet further embodiments at least 90% of the exterior surface of the conductor defines a roughened surface region or regions.

Reference throughout this specification will also be made to the invention providing a surgical implant conductor with the entire exterior surface of the conductor having been roughened. However as indicated above other embodiments with lower proportions of roughened surface areas, or with different distributions of roughened areas are also within the scope of the invention.

A roughened surface region provided with the invention increases the surface area of the conductor compared with that of a non-roughened region. Surface roughness as discussed throughout this specification will be understood by those skilled in the art to be a measurable quality of a surface. This roughness quality can be quantified by measuring the amplitude of a surface's deviations from a normal smooth surface vector. For example, an average roughness Rₐ measurement may be employed to measure roughness from a sum of the absolute value of deviations from the normal vector of the surface. The second quantity used to specify the roughness is the depth of the roughness. For example, if the roughness profile is a square wave with equal mark-space ratio and depression depth equal to the mark or space length, then the increase of surface area is always 3×, and by specifying the roughness and the depth the roughness is fully specified.

Preferably the roughened surface provided by the invention may increase the area of the exterior surface by at least twice that exhibited by a non-roughened surface. In a further preferred embodiment the roughened surface may increase the area of the exterior surface by between 5 to 10 times when compared to that of a non-roughened surface.

The roughened exterior surface of the implant conductor results in a potentially substantial reduction in the effective cross-section area of the conductor used to transport alternating currents. Conversely this surface roughening has a minimal effect on the cross section area of the conductor available to transport direct currents.

This behaviour is known as the skin effect, where the presence of eddy currents in the conductor forces alternating currents to be carried in the outer skin of the conductor. As the frequency of the alternating current increases, the depth at which these currents are transported inside the conductor is reduced. A measurement known as the skin depth of a conductor provides an indication of the depth into the conductor where the alternating current density has fallen to approximately 37%, the skin depth 6 being approximated in conductive materials at radio frequencies by:

δ=\sqrt{2ρ/ωμ}

where ρ is the bulk resistivity of the conductor, ω is the angular frequency of the alternating current and p is the permeability of the conductor.

In a conductor of radius r the skin effect therefore restricts the effective cross section area available to alternating currents to approximately A_{eff}=2πr δ. Conversely the cross section area available to direct currents is πr².

The roughness character of the implant conductor's exterior surface therefore increases the alternating current resistance of the conductor. However as the overall cross section area of the conductor is not significantly impacted by surface roughening, the conductor's direct current resistance is not significantly increased. Furthermore, as the skin depth of a conductor decreases as the frequency of the alternating current increases, radio frequency alternating currents are restricted to the region of the conductor close to the exterior surface and therefore are forced to travel through the roughened surface region.

Reference throughout this specification is also made to measurements or dimensions being provided relative to or in multiples of the skin depth of the surgical implant conductor. Those skilled in the art will appreciate that skin depth is a relative term depending on the frequency of the electromagnetic radiation present in the environment of the conductor. In terms of the present invention and this specification such skin depth references should be interpreted relative to the frequency of energy used by an MRI machine or similar equipment and therefore the skin depth of the conductor when exposed to such energies.

Preferably the roughened surface of the conductor is composed from or incorporates at least one fissure extending into the body or interior of the conductor. In a further preferred embodiment a fissure used to form part of a roughened surface may have a substantially radial or transverse orientation, thereby providing an effective reduction in the radial cross-section area of the conductor available to transport alternating currents.

In a preferred embodiment fissures provided in a roughened surface may extend into the centre of the conductor to a depth which is at least approximately equal to or greater than the skin depth of the conductive material used to form the conductor. This characteristic of the invention therefore significantly degrades the effective radial cross-section area the conductor has available to transport alternating currents.

In a further preferred embodiment the depth of fissures extending into the core of the conductor may be approximately equal to twice the skin depth of the conductive material used. Those skilled in the art will appreciate that the skin depth of a conductor carrying radio frequency alternating currents will be relatively short or small. Therefore, providing fissures extending to twice the skin depth will not have a significant impact on the direct current resistance of the conductor.

Those skilled in the art will appreciate that a range of techniques and manufacturing technologies may be employed to provide a surgical implant conductor in accordance with the present invention. In particular, existing prior art implant conductors of various forms and compositions may be processed by a range of operations to exhibit the roughened surface regions required by the invention.

For example, in some embodiments acid chemical etching, electrochemical and/or electrolysis processes may be used to remove material from the exterior of a conductor to form a roughened surface region. Alternatively, in other instances the manufacturing process of the original conductor may be varied to eliminate any annealing steps. In yet other embodiments mechanical manipulation processes may be used to fatigue and roughen the exterior surface, while in yet other embodiments ablative processes such as plasma etching or laser ablation may be used.

Those skilled in the art will appreciate that these roughening techniques may result in a variety of patterns being applied the exterior surface of the implant conductor. For example, in the case of chemical etching, electrochemical or electrolysis techniques a comparatively random surface patterning may result on the surface of the conductor. Conversely in other embodiments the surface pattern applied may be substantially regular in nature, potentially being provided by the formation of a regular array of fissures to roughen the conductor's exterior surface. Those skilled in the art will appreciate that the roughened surface utilised by the invention may not necessarily exhibit an irregular surface pattern. A range of surface patterns may be applied, provided that the roughened surface increases the exterior surface area, and in preferred embodiments includes fissures which extend at least to the skin depth of the conductive material.

The present invention may therefore provide potential advantages over the prior art or at least provide an alternative choice to the existing prior art.

The invention can be used to mitigate the health risks associated with the exposure of surgical implants to strong electrical fields. The implant conductor provided by the invention can increase the radio frequency alternating current resistance of such conductors without necessarily significantly increasing their direct current resistance.

The implant conductor provided by the invention may be used with a wide variety of prior art implant conductor materials and may also provide safety advantages over a range of frequencies of alternating current. In particular the invention may safeguard surgical implant users from tissue heating and induced current effects in various situations ranging from exposure to MRI scanning machines through to, for example, proximity to electrical welding machines or strong electromagnetic signal transmitters.

### BRIEF DESCRIPTION OF THE DRAWINGS

Additional and further aspects of the present invention will be apparent to the reader from the following description of embodiments, given by way of example only, with reference to the accompanying drawings in which:

FIG. 1 illustrates the heating effect experienced by a conductor exposed to an MRI scanning machine where the conductor has a length close to half the wavelength of that used by the scanning machine, and

FIG. 2a shows a plot of average electric field strength experienced by a surgical implant conductor compared to the length of the conductor measured in wavelengths of the electric field which the conductor is exposed to, and

FIG. 2b shows a series of plots of the change in temperature experienced at the terminal end of a surgical implant conductor when compared with the changes in the length of the conductor measured in wavelengths of the electric field which the conductor is exposed to, and

FIG. 3a provides a side longitudinal section view of a section of a surgical implant conductor provided in accordance with one embodiment of the invention, and

FIG. 3b provides a side longitudinal section view of a section of a surgical implant conductor provided in accordance with a further embodiment of the invention, and

FIG. 4 provides a perspective view of a section of a surgical implant conductor provided in accordance with an alternative embodiment of the invention to that illustrated with respect to FIGS. 3a and 3b

### BEST MODES FOR CARRYING OUT THE INVENTION

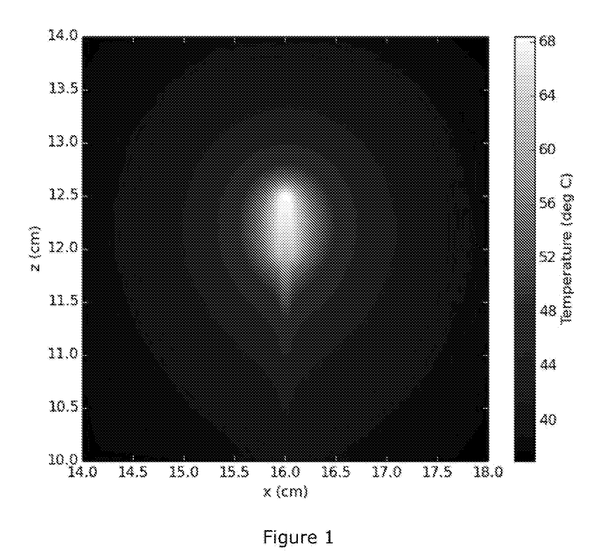

FIG. 1 illustrates the heating effect experienced by a conductor exposed to an MRI scanning machine. In the situation shown the conductor has a length close to half the wavelength of that used by the scanning machine.

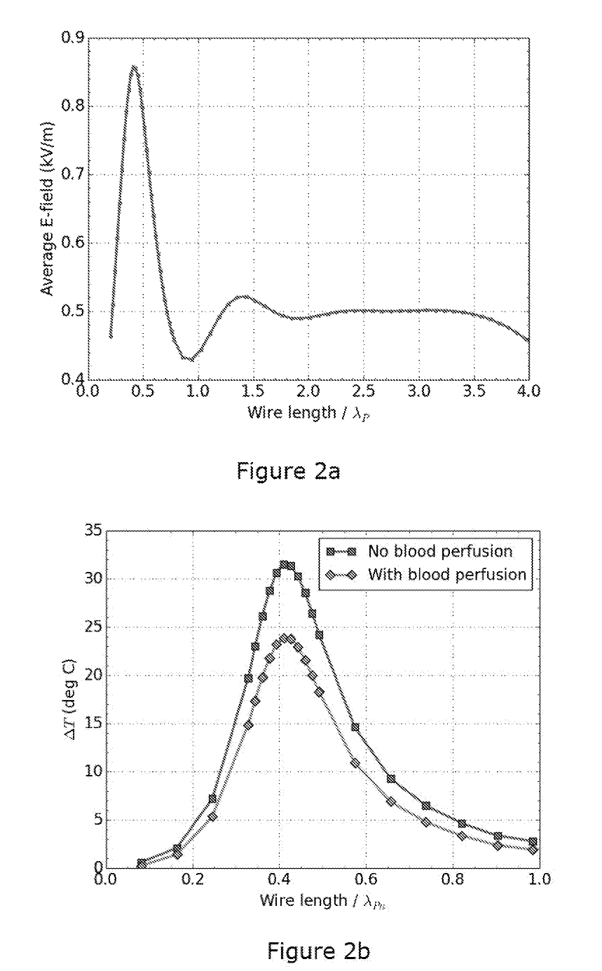

FIG. 2a shows a plot of average electric field strength experienced by a surgical implant conductor compared to the length of the conductor measured in wavelength increments of the electric field which the conductor is exposed to, while FIG. 2b shows a series of plots of the change in temperature experienced at the terminal end of this conductor.

As can be seen by these images there is the potential for significant patient safety issues when users of surgical implants are exposed to MRI scanning machines. Both FIGS. 2a and 2b show how implant conductors of lengths approximating half the wavelength of the MRI machine frequency result in significant concentrations of electric field and associated increases in temperature at the electrode end of these conductors.

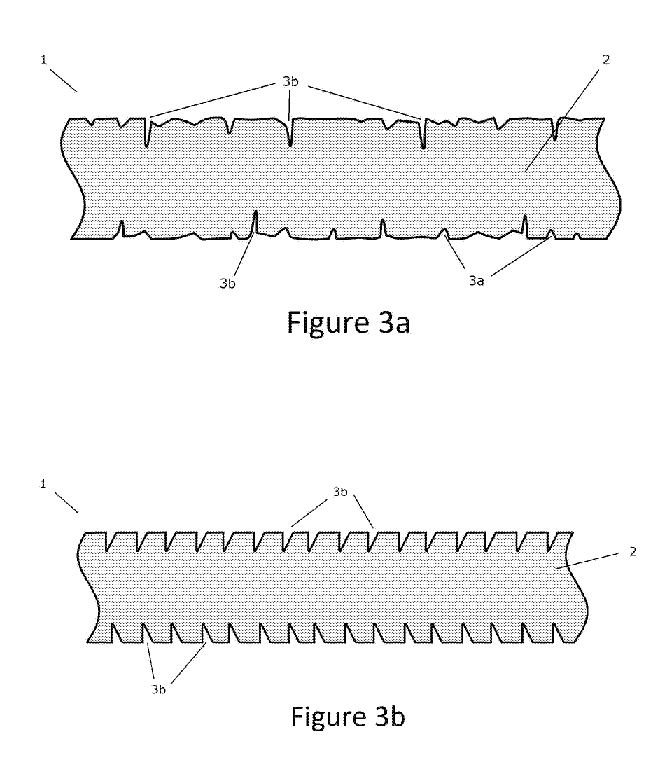

FIG. 3a provides a side longitudinal section view of a section of a surgical implant conductor provided in accordance with one embodiment of the invention. FIG. 3b provides the same view of a surgical implant conductor provided in accordance with a further embodiment.

Each implant conductor 1 is formed from a length of conductive material defining an exterior surface 2. In both the embodiments shown effectively the entire exterior surface of the conductor defines a single region with a roughened surface.

The conductors shown have a substantially uniform cross-section, with the roughening of the exterior surface increasing the area of the exterior surface when compared to a non-roughened surface. In the embodiments shown in FIGS. 2a and 2b this roughening results in a minimum increase of two times that of a non-roughened exterior conductor surface.

Each roughened region defines a number of fissures 3 which extend into the body of the conductor 1. As shown by these figures each fissure has a substantially radial or transverse orientation.

Fissures 3a extends into the body of the conductor to a depth approximately equal to the skin depth of the conductor. Fissures 3b extend to a depth approximately equal to twice the skin depth of the conductor.

As can be seen from a comparison between FIGS. 3a and 3b the roughened surface of the conductor can exhibit a relatively random surface pattern (as with FIG. 3a) or a regular surface pattern (as with FIG. 3b). In both instances the roughened surface increases the area of the exterior surface of the conductor and includes fissures which extend to approximately twice the skin depth of the conductor.

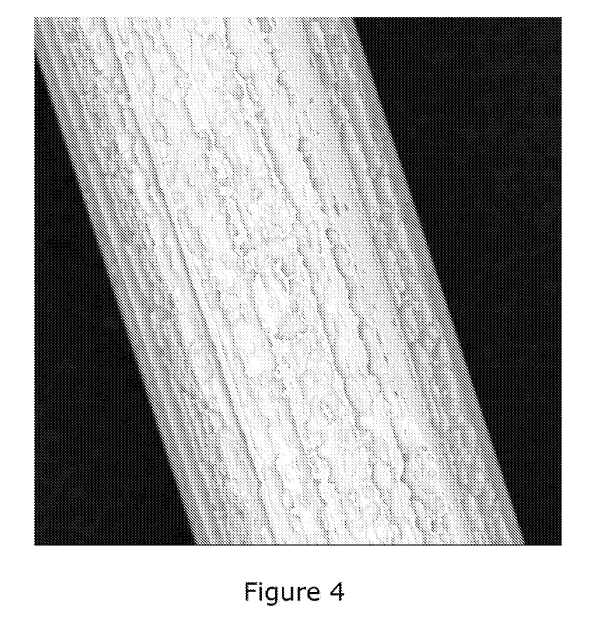

FIG. 4 provides a perspective view of a section of a surgical implant conductor provided in accordance with an alternative embodiment of the invention to that illustrated with respect to FIGS. 3a and 3b. Again it can be seen from this image that the entire exterior surface of the conductor has been roughened. A comparison between FIGS. 3a, 3b and 4 show the results achieved by a variety of manufacturing techniques, from physical manipulation with FIG. 3a, laser ablation with FIG. 3b and acid etching with FIG. 4.

In the preceding description and the following claims the word “comprise” or equivalent variations thereof is used in an inclusive sense to specify the presence of the stated feature or features. This term does not preclude the presence or addition of further features in various embodiments.

It is to be understood that the present invention is not limited to the embodiments described herein and further and additional embodiments within the spirit and scope of the invention will be apparent to the skilled reader from the examples illustrated with reference to the drawings. In particular, the invention may reside in any combination of features described herein, or may reside in alternative embodiments or combinations of these features with known equivalents to given features. Modifications and variations of the example embodiments of the invention discussed above will be apparent to those skilled in the art and may be made without departure of the scope of the invention as defined in the appended claims.

1. A surgical implant conductor with increased radio frequency alternating current resistance formed from a length of conductive material which defines an exterior surface wherein at least a portion of the exterior surface of the conductor defines a region with a roughened surface.

2. A surgical implant conductor as claimed in claim 1 wherein the roughening of the exterior surface increases the area of the exterior surface when compared to a non-roughened surface.

3. A surgical implant conductor as claimed in claim 1 wherein the roughened exterior surface of the implant conductor results in a reduction in the effective cross-section area of the conductor used to transport alternating currents.

4. A surgical implant conductor as claimed in claim 1 wherein a roughened region of the exterior surface results in an increase of at least two times the surface area of the exterior surface when compared with a non-roughened surface.

5. A surgical implant conductor as claimed in claim 1 wherein a roughened region of the exterior surface results in an increase of between five to ten times the surface area of the exterior surface when compared with a non-roughened surface.

6. (canceled)

7. (canceled)

8. A surgical implant conductor as claimed in claim 1 wherein the roughened surface region or regions are covered by an insulating enclosure applied to the conductor.

9. A surgical implant conductor as claimed in claim 1 wherein the roughened surface region or regions are located at or adjacent to the midpoint of the length of the conductor.

10. A surgical implant conductor as claimed in claim 1 wherein at least 50% of the exterior surface of the conductor defines a roughened surface region or regions.

11. A surgical implant conductor as claimed in claim 1 wherein at least 90% of the exterior surface of the conductor defines a roughened surface region or regions.

12. A surgical implant conductor as claimed in claim 1 wherein a roughened region defines at least one fissure extending into the body of the conductor.

13. A surgical implant conductor as claimed in claim 12 wherein a fissure extends into the body of the conductor with a substantially radial or transverse orientation.

14. A surgical implant conductor as claimed in claim 12 wherein a fissure formed in a roughened region extends into the body of the conductor to a depth approximately equal to or greater than the skin depth of the conductor.

15. A surgical implant conductor as claimed in claim 12 wherein the depth of the fissure extending into the core of the conductor is approximately equal to twice the skin depth of the conductive material used.

16. (canceled)

17. (canceled)

18. A method of manufacturing a surgical implant conductor as claimed in claim 1 characterised by the step of roughening at least a portion of the exterior surface of the conductor to define at least one region with a roughened surface.

19. A method of manufacturing a surgical implant conductor as claimed in claim 18 wherein a chemical etching, electrochemical and/or an electrolysis processes is used to remove material from the exterior of a conductor to form a roughened surface region.

20. A method of manufacturing a surgical implant conductor as claimed in claim 18 wherein at least one roughened surface region is formed on the exterior surface of the conductor by eliminating any annealing steps from the conductor manufacturing process.

21. A method of manufacturing a surgical implant conductor as claimed in claim 18 wherein a mechanical manipulation process is used to roughen the exterior of a conductor to form a roughened surface region.

22. A method of manufacturing a surgical implant conductor as claimed in claim 18 wherein an ablative process is used to roughen the exterior of a conductor to form a roughened surface region.

23. A method of manufacturing a surgical implant conductor as claimed in claim 18 wherein a random surface roughening patterning is presented on the surface of the conductor.

24. A method of manufacturing a surgical implant conductor as claimed in claim 18 wherein a regular surface roughening patterning is presented on the surface of the conductor.
